(19) United States
(12) Patent Application Publication
(10) Pub. No.: US 2017/0368365 A1
(43) Pub. Date: Dec. 28, 2017

(54) DEVICE AND METHOD FOR ASSAYING THE APPLICATION OF ENERGY TO SAMPLES IN VITRO

(71) Applicant: ChiScan Holdings, LLC, Las Vegas, NV (US)

(72) Inventors: Bryon K. Eckert, Chandler, AZ (US); Bradley N. Eckert, Chandler, AZ (US); Huan Truong, Chandler, AZ (US)

(21) Appl. No.: 15/635,037

(22) Filed: Jun. 27, 2017

Related U.S. Application Data
(60) Provisional application No. 62/355,543, filed on Jun. 28, 2016.

(51) Int. Cl. A61N 1/40; A61N 1/32; A61N 1/06; C12N 13/00; A61B 5/00; A61N 2/00

(52) U.S. Cl. CPC A61N 1/40; A61N 1/327; A61N 1/06; A61B 5/00; A61N 2/004; C12N 13/00

(57) ABSTRACT

An assay device and method of assaying the application of energy to a plurality of samples in vitro. The assay device includes a housing, an array, and a control module. The housing includes a socket sized to receive a culture plate. The array includes a plurality of emitters and is positioned within the housing adjacent to the socket so that the plurality of emitters are each aligned with at least one culture well of the culture plate when the culture plate is received in the socket. The control module is operably coupled to the array and is configured to individually drive the plurality of emitters. The control module is configured to drive at least two of the plurality of emitters at different frequencies simultaneously.

### RELATED APPLICATIONS

This application claims priority under 35 U.S.C. §119 to U.S. Provisional Patent Application No. 62/355,543 filed on Jun. 28, 2016, the entire contents of which is incorporated herein by reference.

### FIELD OF INVENTION

This invention relates to assaying the application of energy to samples. More specifically, this invention relates to a device for applying various amounts of energy, such as electromagnetic energy, to different wells of a culture plate.

### BACKGROUND

Applying electromagnetic energy to living cells and tissues has been shown to have biological effects on the cells and tissues. Various types of energy have been used for treatment, such as non-thermal plasma, radio frequency, light, electrical fields, and magnetic fields. Pulsing, or modulating, the energy at different frequencies can change the effect of the treatment. However, clinical studies for determining optimum frequencies for specific treatments have been difficult to justify because the physical mechanism behind the biological interaction is not well understood. Clinical studies are also very expensive and the protocol for selecting an optimum frequency is poorly defined.

Conventionally, in vitro studies have been used to determine an optimal dosage of a medicament that causes a desired outcome. In one approach, cells or tissue samples are deposited in a multi-well culture plate and each well is dosed with a different amount of the medicament. The dosage is preferably applied with a programmed robotic device to increase speed and accuracy of the testing. The cells or tissue samples of each well may then be individually analyzed to determine the effects of the applied dosage.

While many methods and devices exist for studying medicament dosages applied to cells or tissue samples, little exist for studying energy applied to cells or tissue samples in an inexpensive and accurate manner. Thus, it would be desirable to have a device for performing in vitro studies with various types of energy.

### SUMMARY

Some embodiments of the invention provide an assay device for a culture plate including a plurality of culture wells. The assay device includes a housing, an array, and a control module. The housing includes a socket sized to receive the culture plate. The array includes a plurality of emitters and is positioned within the housing adjacent to the socket so that the plurality of emitters are each aligned with at least one of the culture wells when the culture plate is received in the socket. The control module is operably coupled to the array and is configured to individually drive the plurality of emitters. The control module is configured to drive at least two of the plurality of emitters at different frequencies simultaneously.

Some embodiments of the invention provide a method of assaying the application of energy to a plurality of samples in vitro. The method includes applying the plurality of samples to individual wells of a culture plate and aligning at least one energy emitter with each individual well of the culture plate. The method also includes applying energy at a separate frequency toward each individual well via each of the at least one energy emitters for a first time period and incubating the wells for a second time period.

### DESCRIPTION OF THE DRAWINGS

FIG. 1 is a perspective view of an assay device according to one embodiment of the disclosure.

FIG. 2 is a schematic diagram of the assay device of FIG. 1.

FIG. 3 is top view of an RF array for use with one embodiment of the assay device.

FIG. 4 is a cross-sectional view of a portion of the RF array of FIG. 3.

FIG. 5 is top view of an electric field array for use with one embodiment of the assay device.

FIG. 6 is a cross-sectional view of a portion of the electric field array of FIG. 5.

FIG. 7 is a cross-sectional view of a portion of a magnetic field array for use with one embodiment of the assay device.

FIG. 8 is a cross-sectional view of a portion of an LED array for use with one embodiment of the assay device.

FIG. 9 is top view of a plasma emitter array for use with one embodiment of the assay device.

FIG. 10 is a cross-sectional view of a portion of the plasma emitter array of FIG. 9.

FIG. 11 is a flow chart of a method of assaying the application of energy to a plurality of samples according to one embodiment of the disclosure.

### DETAILED DESCRIPTION

Before any embodiments of the disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Generally, embodiments of the present disclosure provide an assay device that enables treating multiple wells of a culture plate simultaneously with different pulse frequencies of energy. The device may receive the culture plate in a socket so that the plurality of wells are aligned in close proximity to an array of energy emitters, each of which emits a different pulse frequency simultaneously. This enables many frequencies to be tested simultaneously, speeding up the search for a frequency that provides a desired outcome on samples in the culture wells.

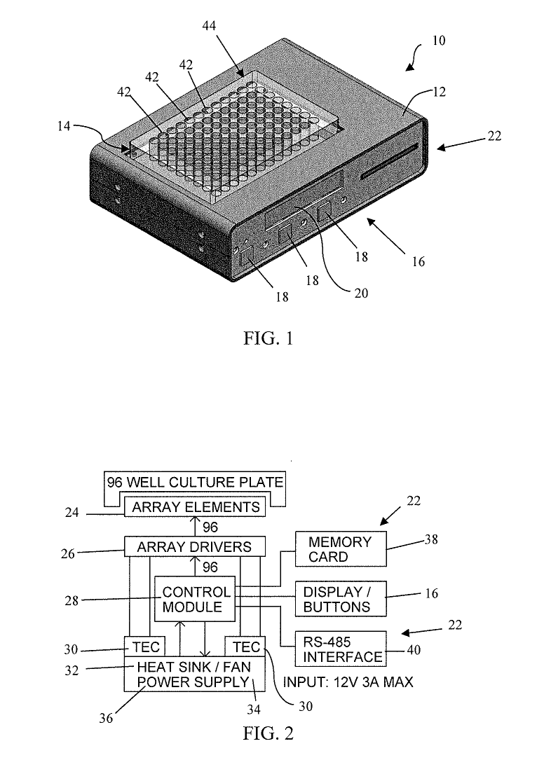

FIG. 1 illustrates an assay device 10 according to some embodiments. As shown in FIG. 1, the assay device 10 can include a housing 12 with a socket 14, a user interface 16 (such as one or more push buttons 18 and a display 20), and one or more input/output interfaces 22. Internally, the device 10 may include an array 24, an array driver 26, a control module 28, one or more thermal control devices 30, a heat sink 32, a fan 34, and a power supply 36, as shown schematically in FIG. 2. Additionally, in one embodiment, as shown in FIG. 2, the input/output interfaces 22 can include a memory card slot 38 and an RS-485 interface 40. As further described below, the assay device 10 is configured to emit energy at different frequencies toward individual wells 42 of a culture plate 44 simultaneously.

As shown in FIG. 1, the socket 14 may be sized to receive a culture plate 44 that includes a plurality of wells 42. For example, the socket 14 may be a square or rectangular cutout in the housing 12 approximately equal to a size of the culture plate 44. It should be noted that, while FIG. 1 illustrates the culture plate 44 with ninety-six wells 42, it is within the scope of this disclosure to include an assay device 10 sized and adapted to receive culture plates 44 including more or less wells 42, such as six wells, twelve wells, twenty-four wells, forty-eight wells, etc. Optionally, the socket 14 may further include a structural support (not shown) around its perimeter to support the culture plate 44. In other embodiments, the culture plate 44 may be received within the socket 14 and rest upon a top outer perimeter of the array 24.

Accordingly, the array 24 can be positioned within the housing 12 adjacent to (e.g., below) the socket 14. As described in more detail below with respect to FIGS. 3-10, the array 24 can include a board 46 with a plurality of energy emitters 48 driven by the array driver 26 to emit energy, such as radio frequency (RF), electric field, magnetic field, optical emission, and non-thermal plasma, toward the culture plate 44. The plurality of emitters 48 may be arranged on the board 46 in an array pattern so that each emitter 48 aligns with at least one culture well 42. As a result, one or more emitters 48 may be dedicated to emit energy at a specific frequency to a single well 42 of the culture plate 44. Generally, in some embodiments, the number of emitters 48 may equal the number of wells 42 for which the device 10 is adapted (such as six, twelve, twenty-four, forty-eight, ninety-six, etc). However, in other embodiments, the array 24 may include more or less emitters 48 than wells 42. For example, the device 10 may be configured so that patterns or groupings of multiple emitters 48 may be dedicated to a single well 42.

According to some embodiments, the control module 28 can be operably coupled to the array 24 (e.g., through the array driver 26) and can be configured to individually drive each emitter 48 at a specific frequency for a set time period. For example, the control module 28 can control the array driver 26 to drive each emitter 48 at a different frequency, or to drive groupings of more than one emitter 48 at different frequencies. The control module 28 may include an application-specific integrated circuit (ASIC), a programmable logic controller (PLC), or, preferably, a field-programmable gate array (FPGA), such as the Xilinx Artix 7. A signal generator for each emitter 48 (e.g., a square wave signal generator) can be implemented in VHDL code as a phase accumulator, or numerically controlled oscillator (NCO). In a preferred embodiment, control module output to the array driver 26 consists of the most significant bit of the phase accumulator. Using only a single bit of the output may add deterministic phase jitter, but it allows the device to define individual emitter units (e.g., up to ninety-six units) with reasonable FPGA resources. Furthermore, using a 200 MHz clock and both edges of the clock, the peak timing uncertainty can be about ±2.5 nanoseconds (ns).

The control module 28 can also be operably coupled (e.g., electrically connected) to the memory card slot 38, the user interface 16, the RS-485 interface 40, the fan 34, the power supply 36 and/or other connections (not shown). More specifically, the memory card slot 38 can be sized to receive a memory device (such as a data card) allowing, for example, the control module 28 to upload control instructions and test data from the memory card and/or download test data to the memory card. Alternatively, in some embodiments, the memory card slot 38 can be sized to receive a USB flash drive. Also, in some embodiments, the control module 28 can include internal memory accessible via one of the I/O interfaces.

In some embodiments, the control module 28 can connect to an external computer via the RS-485 interface 40. The RS-485 interface 40 may also be used to connect multiple devices 10 (e.g., up to 128 devices) in a daisy-chain configuration, for example, using a MODBUS communication protocol. The interface can be designed so that the first device 10 in the cable chain the lowest address, and the subsequent devices 10 have sequentially higher addresses.

In some embodiments, the device 10 can include more than one RS-485 interface 40. Additionally, it should be noted that, while the memory card slot 38 and the RS-485 interface 40 are shown and described herein, it within the scope of this disclosure to include additional input/output interfaces and/or other communications circuits. For example, the device 10 can include a power supply interface (not shown), permitting the device 10 to be connected to and powered by an external power supply.

Accordingly, the control module 28 can receive instructions via the memory card slot 38 (i.e., via an inserted memory device), the RS-485 interface 40 (i.e., via a connected external computer), or additional input/output interfaces. Furthermore, the control module 28 can receive user inputs via the user interface 16. As shown in FIG. 1, the user interface 16 can include one or more push buttons 18. As such, the control module 28 can receive user input via a user pressing one of the push buttons 18. For example, the push buttons 18 can be pressed to control power of the device (i.e., a power button), to toggle between displays or programs (e.g., via “up” and/or “down” buttons), to start a treatment program (e.g., a “start” button), to end a treatment program (e.g., a “stop” button), or for other types of device control. Additionally, in some embodiments, the device 10 can include user inputs other than push buttons, such as rotary dials, switches, or the like.

Furthermore, the user interface 16 can include the display 20. The display 20 may be, for example, a light emitting diode (LED) display, an organic light emitting diode (OLED) display, a digital display, or another suitable display. In one embodiment, the display 20 is a 2×20-character OLED display. The control module 28 may control the display 20 to display to a user, for example, program status, on/off status, device status, present temperatures, a timer, test data, programs, or the like. In one example, the device 20 can display test data or programs in a list format, and a user can toggle the lists and/or select a desired program using the push buttons 18. Alternatively, the control module 28 may automatically execute a program upon receiving a memory card within the memory card slot 38 and being powered on.

Internally, the control module 28 can control operation of the fan 34 as part of a thermal control system of the device 10. More specifically, the thermal control system can include the fan 34, the heat sink 32, the thermal control devices 30 (such as thermoelectric coolers, TECs), and one or more temperature sensors (not shown). For example, the array 24, the array driver 26, and/or the control module 28 can be thermally coupled to the heat sink 32, the TECs 30, and the fan 34. The heat sink 32 may act as a passive cooling device, while the fan 34 may be operated for active cooling, and the TECs 30 may be operated for heating or cooling.

The control module 28 can control operation of the fan 34 and/or the TECs 30 based on a specific treatment program (e.g., the control module 28 may operate the device 10 at a desired temperature or within a desired temperature range based on received temperature control data for a specific program). For example, during and/or after energy application to the culture wells 42, the control module 28 can control the thermal control system (in particular, the fan 34 and the TECs 30) to incubate the culture wells 42 for a preset time period.

More specifically, the temperature sensors can be positioned on the array 24, the housing 12, and/or the heat sink 32 and can be in communication with the control module 28. The control module 28 can monitor the temperatures of these components (i.e., by retrieving a current temperature from one or more of the temperature sensors) and determine whether passive or active cooling is necessary or whether heating is necessary. That is, the control module 28 can operate the fan 34 based on input from one or more of the temperature sensors. In one embodiment, the fan 34 can draw in air from one or more bottom cutouts of the housing 12, past the heat sink 32 and up through the socket 14 (i.e., past the culture plate 44 received in the socket 14). Additionally, in some embodiments, the control module 28 may display an alarm (e.g., via the display 20) if a temperature of one of the components exceeds a high or low temperature threshold for a set time period despite heating or cooling.

Furthermore, it should be noted that other thermal control devices are contemplated within the scope of this disclosure. Additionally, the heat sink 32 and all internal electronics of the device 10 can be thermally insulated from the housing 14.

The following paragraphs describe specific arrays 24 for devices 10 configured to treat culture wells 42 with RF energy, electric fields, magnetic fields, light, and non-thermal plasma, respectively. For example, the above-described components may be common to each device 10, with the exception of the arrays 24 and array drivers 26, which may be specific to the type of energy emission of the device 10. Thus, some embodiments provide different build options for an RF-specific device, an electric field-specific device, a magnetic field-specific device, a light-specific device, and a non-thermal plasma-specific device. Alternatively, in some embodiments, a device 10 can include all the above common components and can be configured to receive different arrays 24 and array drivers 26 in an interchangeable manner. Thus, such a device 10 can be used for all types of energy emissions.

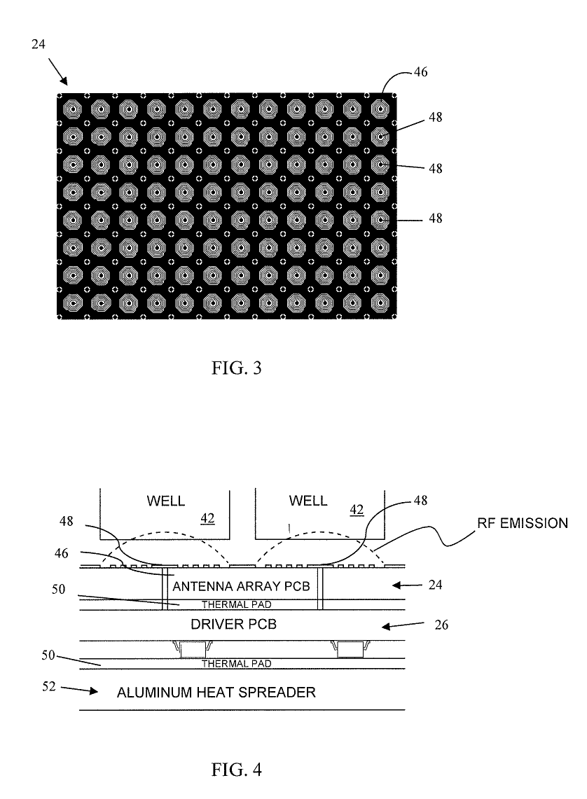

FIGS. 3 and 4 illustrate an RF array 24 according to one embodiment of the disclosure. The RF array 24 comprises an RF emitter board 46 driven by an RF driver board 26. The RF emitter board 46 can be a printed circuit board with rows and columns of individual RF emitters 48. In one embodiment, the RF array 24 can be a 2.4 gigahertz (GHz) array. As such, an RF emitter 48 can be a resonant structure consisting of a printed spiral inductor and series capacitor, which presents a good impedance match at 2.44 GHz. While not an efficient antenna (since most of the power is dissipated in the spiral structure) the emitter 48 is meant to couple short transmissions of RF energy upward and into a culture well 42, similar to that used in near-field communication devices. The matching is relatively narrowband, and the control module 28 can adjust the output RF frequency for each emitter 48 to determine optimum frequencies for certain treatments.

The RF driver board 26 can be a separate printed circuit board that drives the RF array 24, as shown in FIG. 4. A circuit for measuring the forward and reverse power on a single emitter 48 can also be included, on the assumption that all the forward and reverse power on other emitters 48 will be very similar. The RF array driver 26 can include Wilkinson power splitters, RF switches, and load resistors. The control module 28 can route digital signals to the RF driver 26 to create a distinct modulation frequency underneath each culture well 42. The RF array driver 26 can also include a temperature compensated crystal oscillator and frequency synthesizer, as well as amplifiers and attenuators for controlling the RF level. The power into the modulation section can be −10 dBm to +17 dBm, but the maximum radiated power from the array can be about 1 mW. Additionally, as shown in FIG. 4, the RF module (i.e., including the array 24 and the driver 26) can further include thermal pads 50 below each of the array 24 and the driver 26 and a heat spreader 52 below the driver 26. These components can transfer heat through the array driver 26 to the heat sink 32 or other thermal control devices.

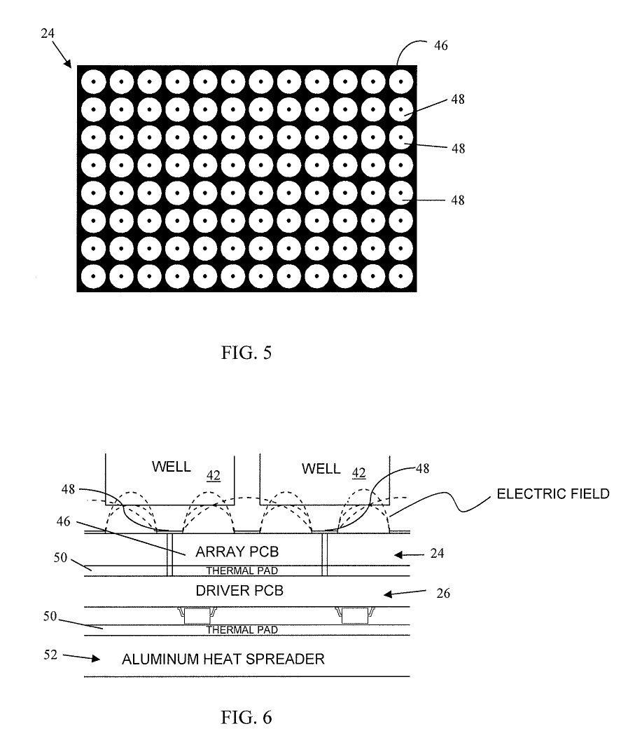

FIGS. 5 and 6 illustrate an electric field array 24 according to one embodiment of the disclosure. The electric field array 24 comprises an electric field emitter board 46 driven by an electric field driver board 26. The electric field emitter board 46 can be a printed circuit board with rows and columns of individual electric field emitters 48. The electric field driver board 26 can be a separate printed circuit board that drives the electric field emitter board 46 to create an electric field flux, as shown in FIG. 6. In one embodiment, for a driving voltage of 5 volts, peak-to-peak (Vp-p), the peak field strength can be between 1.5 volts per centimeter (V/cm) and 2.5 V/cm. In some embodiments, if a higher electric field strength is desired, the array driver voltage can be increased to 190 Vp-p up to 100 kiloHertz (kHz) using high voltage amplifiers. In some embodiments, the emitters 48 can deliver a peak electric field in a culture well 42 between about 60 V/cm and about 100V/cm. In such embodiments, the electric field array driver 26 also includes a voltage boost converter to generate the 200-volt bias for the amplifiers. Additionally, as shown in FIG. 6, the energy field module (i.e., including the array 24 and the driver 26) can further include thermal pads 50 below each of the array 24 and the driver 26 and a heat spreader 52 below the driver 26. These components can transfer heat through the array driver 26 to the heat sink 32 or other thermal control devices.

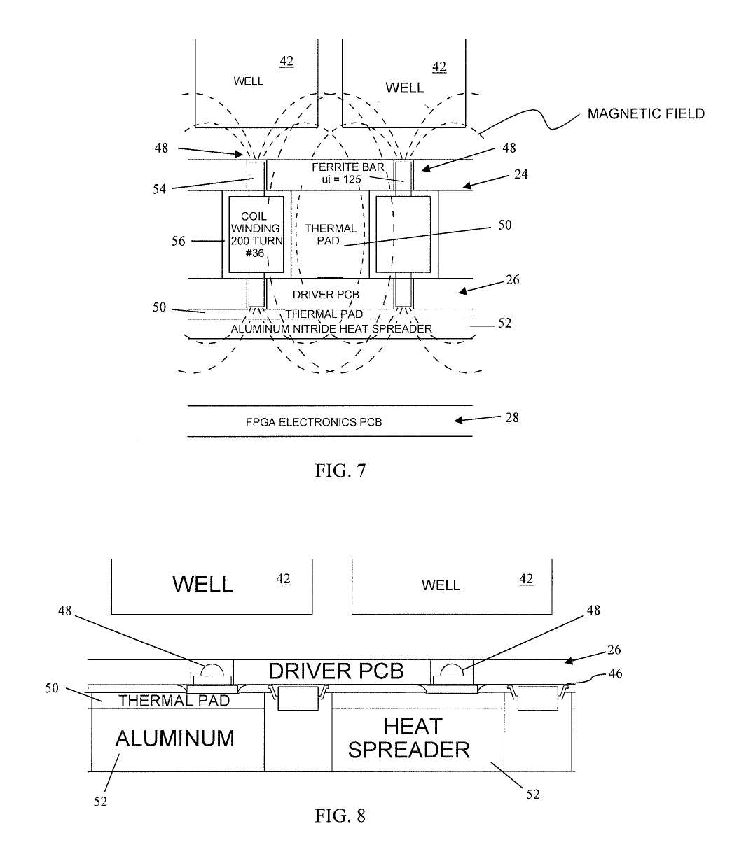

FIG. 7 illustrates a magnetic field array 24 according to one embodiment of the disclosure. The magnetic field array 24 includes a magnetic field emitter board 46 driven by a magnetic field driver board 26. The magnetic field emitter board 46 can be a printed circuit board, functioning mainly to hold the tops of magnetic field emitters 48. More specifically, a magnetic field emitter 48 consists of a ferrite bar 54, which in a preferred embodiment has a length of 7.5 mm and diameter of 0.8 mm. Coil winding 56 around the bar 54 can include 200 turns of #36 wire, presenting about 100 uH of inductance. The moderate permeability (ui=125) of the ferrite bar 24 will tend to efficiently couple higher harmonics generated by the driving waveform.

The magnetic field array driver 26 can be a separate printed circuit board that drives the magnetic field array 24. In some embodiments, the duty cycle of the driving waveform can be adjusted to produce a DC bias of 2 milliamperes (mA) at 5 V. A circuit (not shown) for monitoring DC bias can be connected to a single element 48, on the assumption that all elements 48 will have a similar bias. A calibration routine can be used to determine the required duty cycle for a given frequency. Additionally, as shown in FIG. 7, the magnetic field module (i.e., including the array 24 and the driver 26) can further include thermal pads 50 between the emitters 48 and a heat spread 52 below the magnetic field driver 26, which can conduct heat past the control module 28 over to the TECs 30 (e.g., via aluminum blocks, not shown). In one embodiment, the heat spreader 52 can include aluminum nitride, which will keep metal farther away from the ends of the inductors. Also, in one embodiment, the driver PCB 26 can be positioned to maintain an area around the ferrite bars 54 with no metal for a diameter of about 0.25 inches.

FIG. 8 illustrates an optical array 24 according to one embodiment of the disclosure. The optical array 24 includes an optical emitter board 46 driven by an optical driver board 26. The optical emitter board 46 can be a printed circuit board with light emitting diodes (LEDs) 48 mounted on it. LEDs 48 with a wide variety of wavelengths may be used in certain embodiments, for example from near infrared (NIR, around 850 nanometers (nm)) to ultraviolet (UV, around 395 nm). In one embodiment, the LEDs 48 can be in a 1206 package with lens.

The optical driver board 26 can be a separate printed circuit board that drives the optical array 24. The LED drivers 26 can be designed to run at a fixed current, but the average power can be reduced by adjusting the duty cycle. In some embodiments, the LEDs 48 can be mounted upside down into the driver PCB 26 to create a flush surface for thermal contact with the culture plate 44. Additionally, as shown in FIG. 8, the optical module (i.e., including the array 24 and the driver 26) can further include thermal pads 50 around the LEDs 48 to improve thermal transfer and a heat spreader 52 below the driver 26. These components can transfer heat through the array driver 26 to the heat sink 32 or other thermal control devices.

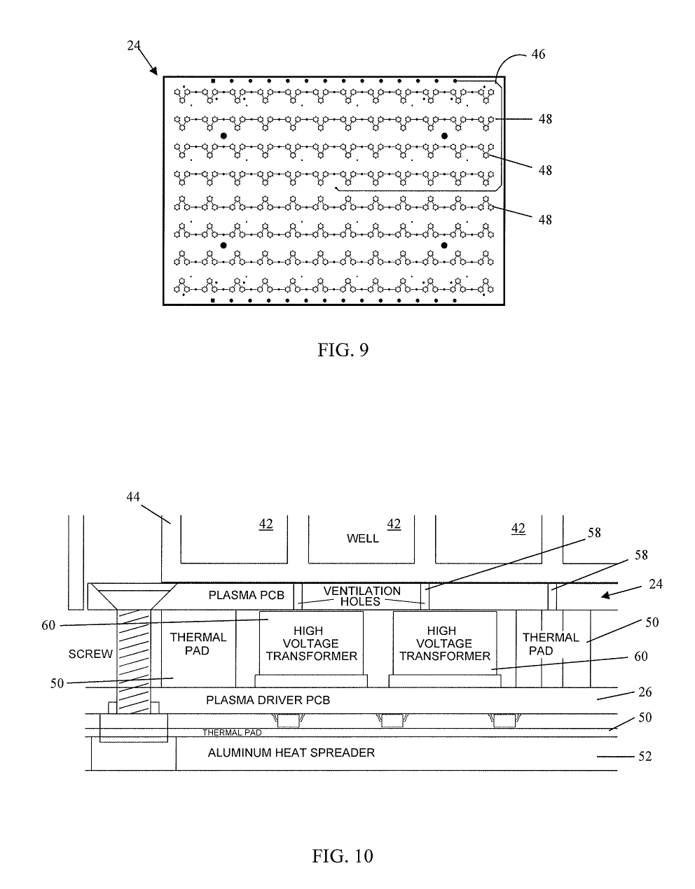

FIGS. 9 and 10 illustrate a plasma array 24 according to one embodiment of the disclosure. The plasma array 24 comprises a plasma emitter board 46 driven by a plasma driver board 26. The plasma emitter board 46 can be a printed circuit board with rows and columns of individual plasma emitters 48. In one embodiment, the plasma emitters 48 can be those described in co-pending U.S. patent application Ser. No. 15/055,028, which is incorporated herein by reference in its entirety. The distance between the plasma dielectric and the base of the culture plate 44 may be defined by a copper and solder mask thickness of about 2 mils. To maintain constant plasma characteristics, ventilation holes 58 may be included outside the plasma areas.

The plasma array driver 26 can be a separate printed circuit board that drives the plasma array 24. In one embodiment, the plasma array driver 26 can require multiple lines from the control module 28 to control the plasma driver 26 and high voltage transformers 60 to create a total of twenty-four drivers 26. Each driver 26 can then be connected to a set of 4 micro-plasma arrays 24, which sit underneath the culture wells 42. Each set of plasma arrays 24 can present a capacitance of about 20 picofarads (pF). This capacitance resonates with the plasma drive transformer 60, and the plasma drive frequency can be about 420 kHz. In one embodiment, the plasma modulation frequency can be adjusted by the control module 28 up to 10 kHz. The plasma tends to run at a fixed current, and a duty cycle of 10% may be used to reduce average power and extend the array life. Additionally, as shown in FIG. 10, the plasma module (i.e., including the array 24 and the driver 26) can further include thermal pads 50 below each of the array 24 and the driver 26 and a heat spreader 52 below the driver 26. These components can transfer heat through the array driver 26 to the heat sink 32 or other thermal control devices.

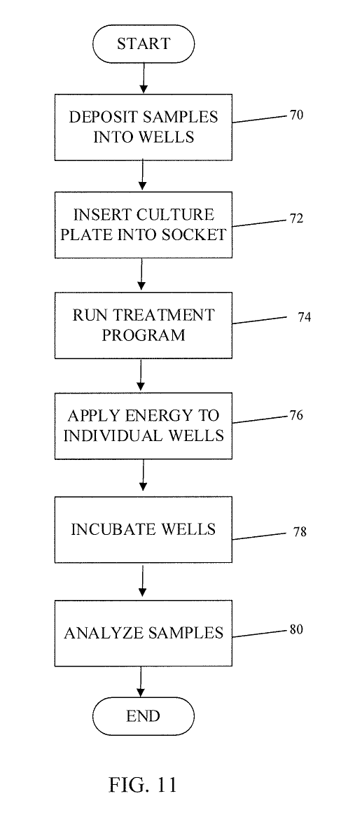

In light of the above, embodiments of the present disclosure provide a device 10 that enables treating multiple wells 42 of a culture plate 44 simultaneously with different pulse frequencies of energy, such as RF, electric field, magnetic field, light, or plasma. FIG. 11 illustrates a method for using the device 10 according to one embodiment of the disclosure. Generally, as shown in FIG. 11, at step 70, a sample culture of interest, such as cells, tissues, bacteria, fungus or virus, is deposited in each well 42 of the culture plate 44. Next, at step 72, the culture plate 44 is inserted into the socket 14 of the device 10. A treatment program is then selected and run, at step 74, and energy at different frequencies is applied to each of the wells simultaneously for a time period, at step 76. During or after the time period in step 76, the culture wells are incubated for a second time period, at step 78. After the time period in step 78, the cultures in each culture well may be analyzed, at step 80, to determine the effects of the applied energy to each well, for example to determine an optimal frequency for a desired outcome.

More specifically, at step 70, a sample culture of interest, such as cells, tissues, bacteria, fungus or virus, is deposited in each well 42 of the culture plate 44. For example, each well may include the same type of sample, or different wells may include different types of samples. Next, at step 72, the culture plate 44 is inserted into the socket 14 of the device 10. As described above, the socket 14 can be sized so that the culture plate 44 rests within the socket 14 atop the array 24. Additionally, in some embodiments, steps 70 and 72 may be reversed (i.e., the culture plate 44 can be inserted into the socket 14 and then the samples can be deposited in each well 42).

When the culture plate 44 is received within the socket 14, a treatment program may be run on the device 10 at step 74. Generally, a treatment program can include specific frequencies to apply, the first set time period, the second set time period, temperature set points or thresholds, and/or other testing parameters. For example, the device 10 may be powered on and a memory card may be inserted in the memory card slot 38 so that the control module 28 automatically executes a treatment program stored on the memory card. In another example, the device 10 may display multiple treatment program options on the display 20 and a user may select a desired treatment program for the control module 28 to run via the push buttons 18. In yet another example, the device 10 may be connected to an external computer via the RS-485 interface 40 (or another input/output interface 22) and a user may select a desired treatment program for the control module 28 to run via the external computer.

According to the selected treatment program, the control module 28 can control the array 26 to apply energy to the culture plate 44 at step 76. More specifically, the control module 28 can generate a set of separate frequencies (i.e., more than one frequency) for each emitter 48 or for groups of emitters 48 to emit toward individual wells 42 of the culture plate 44 simultaneously. As discussed above, the array 24 can be configured so that a single emitter 48 is aligned under each well 42, or so that groups of emitters 48 are aligned under each well 42. Thus, each emitter 48 (or each group of emitters 48) can output a different frequency simultaneously in order to test the effects of multiple frequencies on separate samples (i.e., in the separate culture wells 42) at the same time. In one example, the control module 28 can drive at least two emitters 48 at different frequencies simultaneously. In another example, the control module 28 can generate a set of ninety-six separate frequencies simultaneously to individual control ninety-six emitters 48. The control module 28 can apply the energy to the culture wells 42 for a first set time period according to the selected treatment program.

The control module 28 can also operate the thermal control system to incubate the culture wells 42 for a second set time period, at step 78, according to the selected treatment program. For example, in one embodiment, the control module 28 can thermally control the temperature of the array 24 in order to incubate the wells. In some embodiments, the second set time period may be equal to the first set time period of step 76. Additionally, in some embodiments, the second set time period may partially or completely overlap with the first set time period.

Once the second set time period is completed, the culture wells 42 may be analyzed to determine the effects of the applied energies to the samples, at step 78. In some embodiments, the device 10 may automatically turn off once the second set time period is completed, may automatically save treatment data to memory (e.g., store frequency and temperature control data to internal memory or the memory device), or may display an alert via the display 20 to notify a user that the treatment program is complete.

In accordance with the above method, multiple samples can be simultaneously treated in vitro with different frequencies and examined to determine which sample had a desired or optimal outcome, thus allowing a determination of the optimal frequency for a specific treatment. Furthermore, the method may be repeated, for example, to see if the energy effects are repeatable. For example, the control module 28 can apply the same treatment program, but shuffle the modulation frequencies to different emitters 48. In particular, repeating the methods and shuffling the frequencies can compensate for signal overlap between adjacent array elements. Additionally, the present methods can be applied to multiple devices 10 simultaneously using a daisy-chain configuration, as described above. Multiple tests can thus be completed quickly and accurately and both frequency and temperature data may be stored, for example, to memory devices or a database as part of a research program and to track tested frequencies.

In light of the above, embodiments of the disclosure provide a device and method for studying the application of energy at different frequencies to samples in vitro. By allowing multiple samples to be tested at different frequencies simultaneously and then incubated, the present device and method provide a standalone low-cost, quick, and accurate way to study the biological effects of samples based on the modulation frequency of RF, electric fields, magnetic fields, light, and plasma.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. An assay device for a culture plate including a plurality of culture wells, the assay device comprising:
a housing including a socket sized to receive the culture plate;
an array including a plurality of emitters, the array positioned within the housing adjacent to the socket so that the plurality of emitters are each aligned with at least one of the culture wells when the culture plate is received in the socket; and
a control module operably coupled to the array and configured to individually drive the plurality of emitters, the control module configured to drive at least two of the plurality of emitters at different frequencies simultaneously.

2. The assay device of claim 1, wherein the plurality of emitters are configured to emit one of the following types of energy: radio frequency; electric field; magnetic field; non-thermal plasma; and light.

3. The assay device of claim 1, wherein the array is positioned below the socket.

4. The assay device of claim 1 and further comprising an array driver, wherein the control module is operably coupled to the array via the array driver.

5. The assay device of claim 1 and further comprising a thermal control system, wherein the control module is configured to control the thermal control system based on a temperature of one of the housing, the array, and a heat sink.

6. The assay device of claim 1 and further comprising a heat sink positioned within the housing.

7. The assay device of claim 1 and further comprising a fan positioned within the housing.

8. The assay device of claim 1 and further comprising a user input in communication with the control module.

9. The assay device of claim 8, wherein the user input includes a display and at least one push button.

10. The assay device of claim 1, wherein the housing includes a memory card slot configured to receive a memory device, and the control module is in communication with the memory card slot.

11. A method of assaying the application of energy to a plurality of samples in vitro, the method comprising:
applying the plurality of samples to individual wells of a culture plate;
aligning at least one energy emitter with each individual well of the culture plate;
simultaneously applying energy at a separate frequency toward each individual well via each of the at least one energy emitters for a first time period; and
incubating the wells for a second time period.

12. The method of claim 11 and further comprising providing a device including an array, wherein the at least one energy emitter is positioned on the array; and positioning the culture plate adjacent to the array.

13. The method of claim 12, wherein the step of positioning the culture plate adjacent to the array includes inserting the culture plate into a socket of the device.

14. The method of claim 12 and further comprising monitoring a temperature of the array.

15. The method of claim 12, wherein the step of incubating the wells includes thermally controlling a temperature of the array.

16. The method of claim 11 and further comprising analyzing the samples after the second time period.

17. The method of claim 11, wherein the first time period and the second time period overlap each other.

18. The method of claim 11 and further comprising receiving a treatment program from a memory device, the treatment program including specific frequencies to apply, the first set time period, and the second set time period.

19. The method of claim 11, wherein the step of applying energy includes of applying one of the following types of energy: radio frequency; electric field; magnetic field; non-thermal plasma; and light

20. The method of claim 11 and further comprising storing frequency and temperature data to memory after the step of applying energy.
